(19) United States
(12) Patent Application Publication
(10) Pub. No.: US 2024/0050013 A1
(43) Pub. Date: Feb. 15, 2024

(54) Wearable Devices with Perspiration Measurement Capabilities

(71) Applicant: Apple Inc., Cupertino, CA (US)

(72) Inventors: Sung H. Kim, Mountain View, CA (US); Hyunsoo Ha, Campbell, CA (US); Muhammad Ahmadi, Santa Clara, CA (US); Justin S. Shi, Santa Clara, CA (US)

(21) Appl. No.: 17/988,236

(22) Filed: Nov. 16, 2022

Related U.S. Application Data
(60) Provisional application No. 63/397,013, filed on Aug. 11, 2022.

(51) Int. Cl. A61B 5/28; A61B 5/277; A61B 5/256; A61B 5/00

(52) U.S. Cl. CPC A61B 5/28; A61B 5/277; A61B 5/256; A61B 5/4266

(57) ABSTRACT

Embodiments are directed to devices, systems and methods for determining a perspiration metric of a user. In some embodiments, a device may include a perspiration sensor having first and second electrodes positioned on a skin-facing exterior surface of the device. Capacitance circuitry may measure a capacitance between the electrodes, which may be used to calculate the perspiration metric. In some embodiments, the device defines a cavity such that one or both of the electrodes extend at least partially into the cavity. Other embodiments include a second perspiration sensor, and measurements from the second perspiration sensor may be used in calculating the perspiration metric.

### CROSS-REFERENCE TO RELATED APPLICATIONS

This application claims the benefit under 35 U.S.C. § 119(e) of U.S. Provisional Patent Application No. 63/397,013, filed Aug. 11, 2022, the contents of which are incorporated herein by reference in their entirety.

### FIELD

The described embodiments relate generally to systems and methods for measuring perspiration, and more particularly for measuring perspiration using capacitance measurement circuitry.

### BACKGROUND

Wearable electronic devices are increasingly incorporating sensors that measure physiological information and activity of a user, which in turn may be used to provide a user information relating to their health and fitness. For example, a smartwatch or band worn by a user may be able to detect motion while a user is exercising. The detected motion may be used to derive one or more motion-based metrics, such as step count, step length, speed, or distance traveled. Motion-based metrics may be used in conjunction with physiological measurements, such as a user's heart rate, to provide an estimate of how many calories a user has burned. Collectively, information determined from the sensors of a wearable electronic device may provide a user helpful information in understanding and monitoring aspects of their wellbeing. It may be desirable to add additional functionality to these wearable electronic devices to improve the scope and/or quality of information they can provide.

### SUMMARY

Embodiments described herein are directed to systems, devices, and methods for calculating a perspiration metric of a user. Some embodiments are directed to a method for measuring physiological signals using an electronic device comprising a plurality of electrodes. The method includes, during a perspiration measurement session, connecting a first set of electrodes of the plurality of electrodes to capacitive measurement circuitry, performing a set of capacitance measurements using the first set of electrodes, and calculating a perspiration metric using the set of capacitance measurements. The method further includes, during an electrocardiogram measurement, connecting a second set of electrodes of the plurality of electrodes to electrocardiogram measurement circuitry and performing an electrocardiogram measurement to generate an ECG waveform. The plurality of electrodes comprises a first electrode and a second electrode, such that the first set of electrodes includes the first electrode and the second electrode and the second set of electrodes includes the first electrode.

In some variations, the second set of electrodes also includes the second electrode. In some of these variations, the plurality of electrodes includes a third electrode and the second set of electrodes includes the third electrode. The perspiration may be an estimated sweat rate or another perspiration metric as described herein. Additionally or alternatively, the perspiration measurement session is initiated while the electronic device is tracking an exercise session. In some of these instances, the perspiration measurement session is initiated automatically in response to the electronic device tracking the exercise session.

Other embodiments are directed to a device for calculating a perspiration metric of a user, where the device includes a housing having a skin-facing exterior surface, a perspiration sensor that includes a first electrode and a second electrode, and a processer configured to calculate the perspiration metric using the perspiration sensor. The skin-facing exterior surface of the housing defines a cavity, and the first electrode extends at least partially into the cavity. In some variations, the second electrode also extends at least partially into the cavity. Additionally or alternatively, a portion of the first electrode is positioned outside of the cavity.

In some variations, the skin-facing exterior surface includes a first region surrounding a second region such that a first portion of the second region is recessed relative to the second region to define the cavity. In some of these variations, a second portion of the second region protrudes past the first region. Additionally or alternatively, the cavity has an annular shape. In some variations, the device includes an optical sensor comprising a set of windows, wherein the set of windows is positioned in the second region. Additionally or alternatively, the perspiration sensor is a first perspiration sensor, and the device further includes a second perspiration sensor. In some instances, the perspiration sensor includes capacitance measurement circuitry configured to measure a capacitance between the first electrode and the second electrode.

Yet other embodiments are directed to a device for calculating a perspiration metric of a user, and that includes a housing having a skin-facing exterior surface that defines a first cavity and a first perspiration sensor comprising a first electrode defining a first portion of the skin-facing exterior surface and a second electrode defining a second portion of the skin-facing exterior surface. The device further includes a second perspiration sensor contained within the first cavity, and a processer configured to calculate the perspiration metric using both the first perspiration sensor and the second perspiration sensor.

In some of these variations, the housing defines a second cavity, and the first electrode extends at least partially into the second cavity. In some of these variations, the second cavity encircles the first cavity. In other variations, the first cavity is positioned between at least a portion of the first electrode and at least a portion of the second electrode. Additionally or alternatively, the first cavity is positioned closer to a center of the skin-facing exterior surface of the housing relative to the first electrode.

In addition to the example aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIGS. 1A and 1B show front and rear views, respectively, of a wearable electronic device having a perspiration sensor as described herein. FIG. 1C shows example capacitance measurement circuitry that may be used with the perspiration sensor of FIGS. 1A and 1B. FIG. 1D shows additional components that may be incorporated into the wearable electronic device of FIGS. 1A and 1B.

FIG. 2 shows an example method of calculating a perspiration metric as described herein.

FIG. 3A shows a hybrid sensor that may selectively be used to generate an electrocardiogram waveform or calculate a perspiration metric. FIG. 3B shows a process flow for performing measurements using the hybrid sensor of FIG. 3A.

FIGS. 4A and 4B show front and side views, respectively, of an example variation of an electronic device having a perspiration sensor as described herein.

FIGS. 5A and 5B show front and side views, respectively, of an example variation of an electronic device having a perspiration sensor and an accessory component as described herein.

FIGS. 6A and 6B show front and side views, respectively, of another variation of an electronic device having a plurality of perspiration sensors as described herein.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to devices, systems, and methods for calculating a perspiration metric of a user. In some embodiments, a device may include a perspiration sensor having first and second electrodes positioned on a skin-facing exterior surface of a housing of the device. Capacitance measurement circuitry may measure a capacitance between the electrodes, which may be used to calculate the perspiration metric. In some embodiments, the device (alone or in combination with a removable accessory) defines a cavity such that one or both of the electrodes extend at least partially into the cavity. Other embodiments include a second perspiration sensor, and measurements from the second perspiration sensor may be used in calculating the perspiration metric.

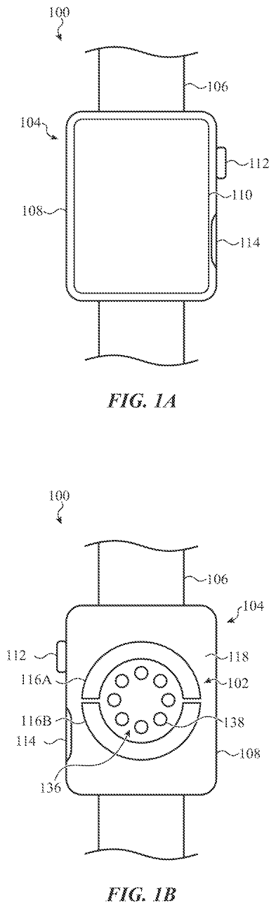

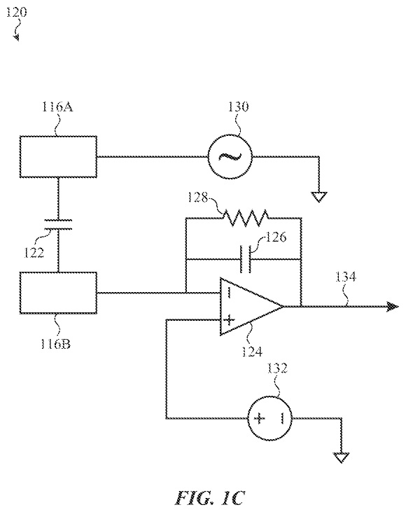

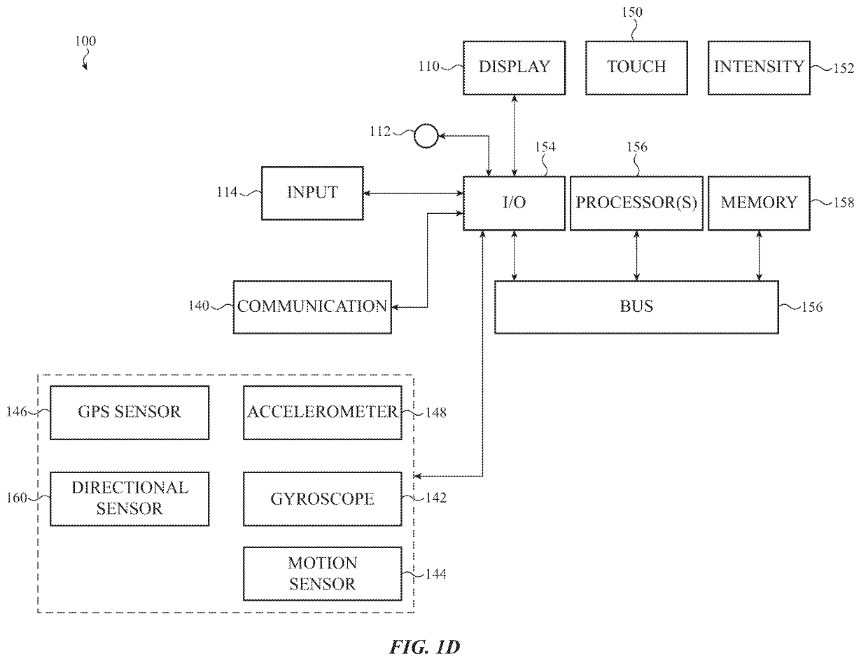

These and other embodiments are discussed below with reference to FIGS. 1A-6B. However, those skilled in the art will readily appreciate that the detailed description given herein with respect to these Figures is for explanatory purposes only and should not be construed as limiting.

The devices, systems, and methods described herein are configured to perform perspiration measurements using one or more perspiration sensors. These measurements are in turn used to calculate an estimated sweat rate of a user and/or additional metrics that utilize a user's estimated sweat rate. Specifically, the estimated sweat rate of a user is a measure of the amount of fluid lost over an interval of time. In instances where an estimated sweat rate is presented to a user, it may be presented in a number of different ways. In some instances the estimated sweat rate may be presented as a rate of fluid loss (e.g., expressed in fluid ounces per hour, or the like) or as an amount of fluid loss for a specific period of time (e.g., expressed as a volume of fluid).

For example, when the devices described herein are used to track an exercise session (e.g., while a user is engaged in a physical activity such as walking, running, cycling, or the like), the device may calculate and present a first estimated sweat rate over a first interval of time corresponding to the exercise session. This may provide a user with information about the average sweat rate or total fluid loss over the course of the exercise session. Additionally or alternatively, the device may calculate and present a second estimated sweat rate over a second interval of time shorter than the first interval of time (i.e., shorter than the entire duration of the exercise session). This second interval of time may be relatively short (e.g., the last five seconds, the last 15 seconds, the last 30 seconds, or the like), and may represent an “instantaneous” sweat rate of the user. In this way, a user may be able to understand their current rate of fluid loss at any given point within the exercise session, as well as their total fluid loss during the exercise session.

Because the measurements performed by a perspiration sensor (or sensors) of a device are taken at a particular location, the measurement is indicative of the perspiration at that particular location (e.g., measurements taken at the wrist of a user reflects how much the user is perspiring at the user's wrist). The devices, systems, and methods may be used to calculate a local estimated sweat rate, which represents an estimate of the sweat rate at the measurement site where the perspiration measurements are performed. Accordingly, this local estimated sweat rate may be converted into a global estimated sweat rate, which represents an indication of the overall sweat rate across the user's entire body. This may provide an estimate of the overall amount of fluid lost by the user's body (e.g., during an exercise session). In these instances, a predetermined relationship between the local estimated sweat rate and the global estimated sweat rate (which may optionally be based on other inputs as discussed below) may be used to convert the local estimated sweat rate to a global estimated sweat rate. Unless otherwise specified, when the systems, devices, and methods are discussed herein as calculating, using, or outputting an “estimated sweat rate” this may be either a local estimated sweat rate or a global estimated sweat rate.

An estimated sweat rate may also be used to calculate one or more additional metrics. In some instances, the estimated sweat rate may be used in calculating a metric indicative of a user's hydration (e.g., a hydration level, a change in hydration of an interval of time, or the like). In other instances, the estimated sweat rate may be used in calculating an exercise metric, such as workout intensity, calories burned, or the like. While some or all of these exercise metrics may be calculated without a perspiration measurement, the use of the estimated sweat rate as an input may improve the accuracy of these measurements. The estimated sweat rate, as well as any additional metric calculated using the estimated sweat rate (such as those described above), are collectively referred to herein as “perspiration metrics.”

The devices and methods described herein may utilize additional measurements in calculating a perspiration metric, which may help improve the accuracy of the calculated perspiration metrics. In some instances, ambient temperature and/or humidity may be used in calculating a perspiration metric. For example, in instances where a local estimated sweat rate is converted to a global estimated sweat rate, the conversion may utilize this ambient temperature and/or humidity information. The overall amount that a user perspires may be dependent on the ambient temperature and/or humidity, and thus the relationship between the local estimated sweat rate and the global estimated sweat rate may also depend on the ambient temperature and/or humidity.

Additionally or alternatively, a body temperature measurement may be used in calculating a perspiration metric. For example, a body temperature measurement may be used in converting a local estimated sweat rate to a global estimated sweat rate. It should be appreciated that any of these additional measurements (e.g., ambient temperature, ambient humidity, body temperature), may be performed by the same device that contains the perspiration sensor or may be performed by a separate device. In some instances, the devices and methods described herein may utilize location information (e.g., from a GPS system and/or from user input) to obtain an ambient temperature and/or humidity value associated with a location of the user.

Generally, the perspiration sensors described herein may be placed against a user's skin to perform the measurements that are used to calculate a perspiration metric. In some instances, a perspiration sensor may be incorporated into a wearable electronic device that may be configured to be releasably coupled to a portion of a user's body. For example, FIGS. 1A and 1B show front and rear views, respectively, of a variation of a wearable electronic device 100 that incorporates a perspiration sensor 102. In the variation shown in FIGS. 1A and 1B, the wearable electronic device 100 is configured as a watch having a watch body 104 and a strap 106. The strap 106 may be attached to the watch body 104 (either releasably, thereby allowing the watch body 104 to be used with different straps, or permanently), and may at least partially encircle a portion of a user's body (e.g., the user's wrist) to releasably couple the wearable electronic device 100 to the user. In other variations, the wearable electronic device 100 may be configured as a band, bracelet, or ring having a housing configured to at least partially encircle a portion of a user's body to releasably couple the wearable electronic device 100 to the user. In still other variations, the wearable electronic device 100 may be releasably coupled to a user via an adhesive.

The wearable electronic device 100 may include a housing 108. In instances where the wearable electronic device 100 is configured as a watch, the housing 108 may be a housing for the watch body 104. The housing 108 defines one or more exterior surfaces of the wearable electronic device 100, and may be formed from a single housing member or formed from two or more housing members that are attached to each other (as will be described in more detail below). Each housing member of the housing 108 may be formed from any suitable material or combination or materials, such as one or more metals, plastics, ceramics, glasses, crystals, or the like.

In some variations, the wearable electronic device 100 includes a display 110. The display 110 may be at least partially positioned within the housing 108, and in some instances includes a transparent cover (e.g., a glass) that protects components of the display 110 positioned within the housing 108. In these instances, the display 110 forms a portion of an exterior surface of the wearable electronic device 100. In some instances, such as shown in FIG. 1A, the display 110 is positioned on (and thereby defines) a front exterior surface of the wearable electronic device 100 such that the display 110 provides a graphical output that is viewable through or at the front exterior surface of the wearable electronic device 100. The wearable electronic device 100 may be configured such that the display 110 is positioned such that it is visible to a user while releasably coupled to the user.

The display 110 may include a display portion that can be implemented with any suitable technology, including, but not limited to liquid crystal display (LCD) technology, light emitting diode (LED) technology, organic light-emitting display (OLED) technology, organic electroluminescence (OEL) technology, electronic paper (e.g., electronic ink) display technology, or another type of display technology. In some instances the display 110 is configured as a touchscreen, and is capable of receiving touch inputs from a user. In these instances, the display 110 may include one or more sensors (e.g., capacitive touch sensors, ultrasonic sensors, or other touch sensors) positioned above, below, or integrated within other components of the display 110.

In some instances, the wearable electronic device 100 may include at least one input mechanism, such as a crown, scroll wheel, knob, dial, button, or the like. A user may manipulate an input mechanism (e.g., by rotating a scroll wheel or pressing a button) to provide an input to the wearable electronic device 100, which may in turn be used to control the wearable electronic device 100 (e.g., to manipulate or select various graphics displayed by the display 110, to adjust a volume of a speaker, to turn the wearable electronic device 100 on or off, or the like). In the variation shown in FIGS. 1A and 1B, the watch body 104 includes a crown 112 and a button 114. The crown 112 may extend at least partially through the housing 108 to allow a user to manipulate the crown 112 (e.g., by rotating or pressing on the crown 112) to provide inputs via the crown 112. Similarly, the button 114 may extend at least partially through the housing 108, and may be depressed by a user to provide an input via the button 114.

As mentioned above, the wearable electronic device 100 includes a perspiration sensor 102. The perspiration sensor 102 includes a first electrode 116A and a second electrode 116B that form at least a portion of an exterior surface of the wearable electronic device 100. For example, as shown in FIG. 1B, the first electrode 116A and the second electrode 116B are positioned on a skin-facing exterior surface 118 of the wearable electronic device 100 (e.g., a skin-facing surface of the watch body 104). Accordingly, when the wearable electronic device 100 is releasably coupled to a user as discussed above, at least a portion of the first electrode 116A and the second electrode 116B may contact the user's skin. In instances where the wearable electronic device 100 includes a display 110, the display 110 may be positioned on a front exterior surface of the wearable electronic device 100 that is opposite the skin-facing exterior surface 118. In the variation shown in FIGS. 1A and 1B, the display 110 and the electrodes of the perspiration sensor 102 are on opposite sides of the watch body 104.

To perform a measurement using the perspiration sensor 102, the first electrode 116A and the second electrode 116B are connected to capacitance measurement circuitry that measures a capacitance between the first and second electrodes 116A, 116B. When the skin-facing exterior surface 118 is positioned against a user's skin, the capacitance measured by the capacitance measurement circuitry may vary depending on the amount of moisture present, and thus may be used to calculate one or more perspiration metrics, as will be described in more detail below. FIG. 1C shows an example capacitance measurement circuitry 120 that may be used to measure a capacitance (illustrated as capacitance 122) between the first and second electrodes 116A, 116B. The capacitance measurement circuitry 120 includes an operational amplifier 124 having an inverting input, a non-inverting input, and an output 134, a feedback resistor 126, a feedback capacitor 128, an input voltage source 130, and a reference voltage source 132.

The input voltage source 130, which is shown in FIG. 1C as being an AC voltage source, is connected to the first electrode 116A. The second electrode 116B is connected to the inverting input of the operational amplifier 124, while the reference voltage source 132 (which is shown in FIG. 1C as being a DC voltage source) is connected to the non-inverting input of the operational amplifier 124. The output 134 of the operational amplifier 124 is coupled back to the inverting input through the feedback resistor 126 and feedback capacitor 128. The output 134 of the operational amplifier 124 may vary with changes in voltage of the input voltage source 130, but also with change in the capacitance 122 between the first electrode 116A and the second electrode 116B. Accordingly, the capacitance 122 may be calculated (e.g., using demodulation or other techniques) from the output 134 of the operational amplifier 124. It should be appreciated that the capacitance measurement circuitry 120 of FIG. 1C is just one example of circuitry that may be used to measure a capacitance between two electrodes, and there are many other configurations of capacitance measurement circuitries that may be utilized to measure a capacitance between the first and second electrodes 116A, 116B.

Returning to FIG. 1B, in some variations the wearable electronic device 100 includes one or more additional sensors configured to measure one or more physiological systems. For example, the wearable electronic device 100 may include an optical sensor 136. The optical sensor 136 includes a set of windows 138 that allow light to be transmitted therethrough. The optical sensor 136 further includes a set of emitters (not shown) positioned within the housing 108 and configured to emit light through at least some of the windows in the set of windows. Similarly, the optical sensor includes a set of light detectors (not shown) positioned within the housing 108 and configured to receive light through at least some of the windows of the set of windows. The set of emitters may direct light from the wearable electronic device 100 toward a user, while the set of detectors may detect light returned from the user to the wearable electronic device 100. The amount of light detected by the set of detectors may be used to calculate one or more physiological signals (such as heart rate, blood oxygenation, or the like) and/or determine a proximity between the wearable electronic device 100 and the skin of the user.

FIG. 1D depicts additional exemplary components of the wearable electronic device 100. In some embodiments, the wearable electronic device 100 has a bus 156 that operatively couples I/O section 154 with one or more computer processors 156 and memory 158. I/O section 154 can be connected to display 110, which can have touch-sensitive component 150 and, optionally, intensity sensor 152 (e.g., contact intensity sensor). In addition, I/O section 154 can be connected with communication unit 140 for receiving application and operating system data, using Wi-Fi, Bluetooth, near field communication (NFC), cellular, and/or other wireless communication techniques. The wearable electronic device 100 can include input mechanisms, such as a crown 112 and/or button 114 as discussed previously. The wearable electronic device 100 optionally includes various sensors, such as GPS sensor 146, accelerometer 148, directional sensor 160 (e.g., compass), gyroscope 142, motion sensor 144, and/or a combination thereof, all of which can be operatively connected to I/O section 154.

Memory 158 of device 100 can include one or more non-transitory computer-readable storage mediums, for storing computer-executable instructions, which, when executed by one or more computer processors 156, for example, can cause the computer processors to perform the techniques that are described herein (e.g., calculating perspiration metrics). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like.

The processor 156 can include, for example, dedicated hardware as defined herein, a computing device as defined herein, a processor, a microprocessor, a programmable logic array (PLA), a programmable array logic (PAL), a generic array logic (GAL), a complex programmable logic device (CPLD), an application-specific integrated circuit (ASIC), a field-programmable gate array (FPGA), or any other programmable logic device (PLD) configurable to execute an operating system and applications of device 100, as well as to facilitate setting a field of view of a camera and capturing of images as described herein. The wearable electronic device 100 is not limited to the components and configuration of FIG. 1D, but can include other or additional components in multiple configurations.

As mentioned above, the perspiration sensor 102 may calculate one or more perspiration metrics using the first electrode 116A, second electrode 116B, and capacitance measurement circuitry (such as the capacitance measurement circuitry 120 of FIG. 1C). For example, FIG. 2 shows a method 200 of calculating a perspiration metric using an electronic device having a perspiration sensor positioned on a skin-facing surface of the electronic device. At step 202, a skin-facing surface of the electronic device is positioned against the skin of a user such that at least a portion of the skin-facing surface contacts the user's skin (though it should be appreciated that in some instances user movement, such as from exercise, may cause momentary separation between the skin-facing surface of the electronic device and the user's skin). In instances where the electronic device is the wearable electronic device 100 of FIGS. 1A-1D, this step may include releasably coupling the watch body 104 to the user using the strap 106, such that at least a portion of the skin-facing exterior surface 118 of the watch body 104 contacts the user's skin. In instances where the wearable electronic device 100 is configured as a watch, the wearable electronic device 100 may be coupled to the wrist. It should be appreciated that the devices described herein may be releasably coupled to different portions of the body (e.g., other locations along a user's arm, their leg, torso, or the like).

At step 204, a perspiration sensor (such as perspiration sensor 102 shown in FIG. 1B) is used to obtain a set of capacitance measurements. For each measurement, a capacitance is measured between first and second electrodes of the perspiration sensor that are positioned on a skin-facing exterior surface of the electronic device. For example, a capacitance measurement circuitry (such as the capacitance measurement circuitry 120 of FIG. 1C) may measure the capacitance between the first and second electrodes (e.g., first and second electrodes 116A, 116B of the perspiration sensor 102 shown in FIG. 1B) to measure the capacitance. The set of capacitance measurements may be performed over a predetermined measurement window. In some instances, the set of capacitance measurements is a single measurement that measures the capacitance between the first and second electrodes continuously during the measurement window. In other instances, the set of capacitance measurements include multiple individual measurements that measure the capacitance at different times within the measurement window. Each capacitance measurement may output one or multiple capacitance values depending on the duration of the individual measurement. Accordingly, the set of capacitance measurements may output a corresponding set of capacitance values.

The set of capacitance measurements may be captured on demand (e.g., in response to a request from a user to determine a perspiration metric), automatically on a scheduled basis (e.g., performed at predetermined times during the day), and/or automatically when certain criteria are met. For example, the set of capacitance measurements may be captured automatically while a device is tracking an exercise session (e.g., to provide perspiration metrics associated with the activity).

At step 206, a perspiration metric is calculated using the set of capacitance measurements (e.g., using a processor of the electronic device). Specifically, the set of capacitance values obtained by the set of capacitance measurements may be analyzed to determine a perspiration metric. In instances where the perspiration metric is an estimated sweat rate, a predetermined relationship between measured capacitance and sweat rate may be used to convert the set of capacitance values to a value of the estimated sweat rate. Similarity, the set of capacitance measurements may be used as an input in determining other perspiration metrics, such as those discussed above. In some variations, the perspiration metric is calculated using one or more additional inputs, including (but not limited to) ambient temperature, ambient humidity, user temperature, physical parameters of the user such as height, weight, or body composition, information from additional sensors, combinations thereof, or the like.

At step 208, the calculated perspiration metric may be outputted. In some instances, this includes storing the perspiration metric in permanent memory (such as a database used to store physiological data of user), which may allow the perspiration metric to later be accessed by the user or another device or application (e.g., a software program on the wearable electronic device 100) as permitted by the user. Additionally or alternatively, the electronic device may output the calculated perspiration metric to a user. This output may be achieved in a variety of ways including through a user interface (U/I) generated as a display (e.g., display 110), using audio alerts, through haptic feedback, or any other suitable output mechanism, or combinations thereof.

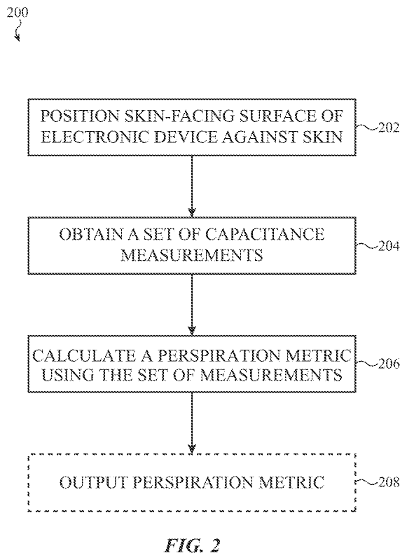

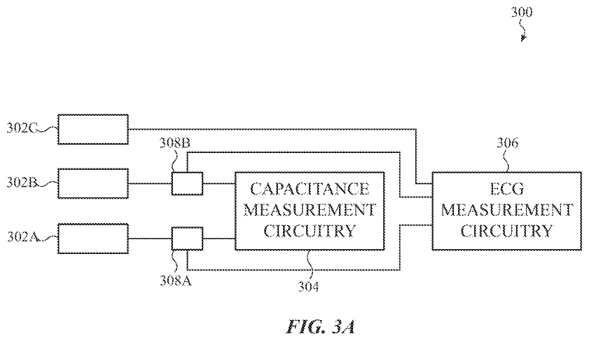

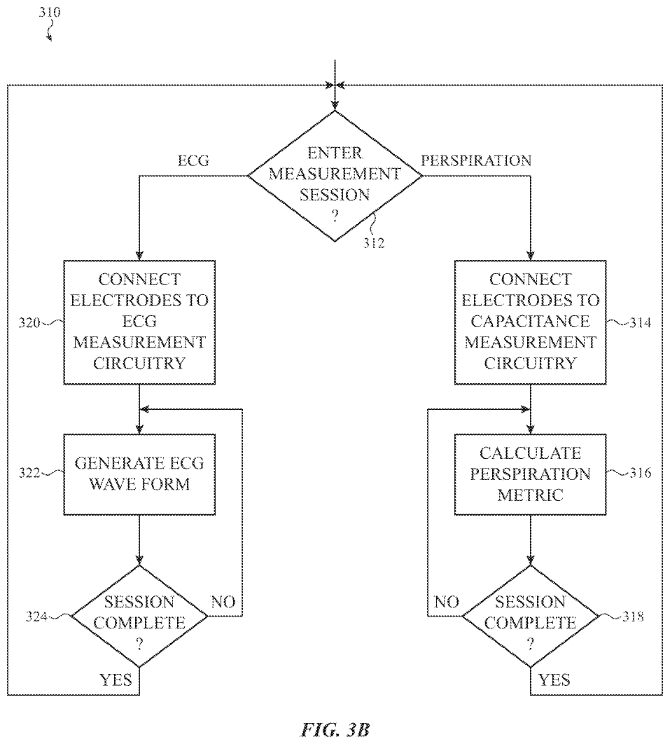

In some instances, one or more electrodes of a perspiration sensor may be also be used to perform additional physiological measurements. For example, in some variations, one or more electrodes of the perspiration sensor may be used to measure an electrocardiogram (ECG) waveform of a user (an “ECG measurement”). FIG. 3A shows an example of a hybrid sensor 300 that may be used to selectively measure an ECG waveform of a user or a perspiration metric of the user. The hybrid sensor 300 includes a plurality of electrodes, including a first electrode 302A, a second electrode 302B, and a third electrode 302C. Different subsets of the plurality of electrodes may be used to perform ECG measurements and perspiration measurements. This allows the same electrode or electrodes to be utilized for multiple different measurements, which may result in space saving and/or reduced cost while still providing the ability of the electronic device to perform this dual functionality.

The hybrid sensor 300 may be part of an electronic device such as the wearable electronic device 100 of FIGS. 1A-1C. For example, the first and second electrodes 302A, 302B may be positioned on (and thereby define) a portion of a skin-facing surface of an electronic device (such as the wearable electronic device 100 of FIGS. 1A-1D), and one or both of these electrodes are positioned to at least partially contact a user's skin when the electronic device is releasably coupled to the user. The first and second electrodes 302A, 302B may be selectively connected to capacitance measurement circuitry 304 (which may be configured in any suitable manner such as discussed above with respect to the capacitance measurement circuitry 120 of FIG. 1C). When the first and second electrodes 302A, 302B are connected to the capacitance measurement circuitry 304, the capacitance measurement circuitry 304 may measure a capacitance between first and second electrodes 302A, 302B, and may use the measured capacitance to calculate one or more perspiration metrics as described herein.

At other times, the first electrode 302A and/or second electrode 302B may be connected to ECG measurement circuitry 306 configured to perform an ECG measurement. In these instances, a third electrode 302C is also connected to the ECG measurement circuitry 306. The ECG measurement circuitry 306 may measure a voltage between the third electrode 302C (referred to as the “measurement electrode” for ease of discussion) and either the first electrode 302A or the second electrode 302B (referred to as the “reference electrode” for ease of discussion), and this voltage may be processed to generate one or more ECG waveforms representing the electrical activity of the heart. It should be appreciated that the ECG measurement circuitry 306 may be any suitable known ECG measurement circuitry as will be readily understood by one of ordinary skill in the art.

To generate the ECG waveform, the measurement electrode and the reference electrode are both placed in contact with user's skin, preferable on different limbs or regions of the user's body. For example, the measurement electrode may be placed in contact with a first arm (e.g., at a wrist) or leg (e.g., at an ankle) and the reference electrode may be placed in contact with a different arm or leg. When the electronic device is releasably coupled to a user, the reference electrode is positioned on the skin-facing surface of the electronic device such that at least a portion of the reference electrode contacts the user. The third electrode 302C is positioned on a portion of an electronic device (e.g., on a portion of a housing of the electronic device, on an input mechanism such as a crown 112 or button 114 described previously, as part of the display of the electronic device, or the like) that allows it to be touched by a different portion of the user.

In some variations, such as shown in FIG. 3A, both the first electrode 302A and second electrode 302B are connected to and used by the ECG measurement circuitry 306 to generate one or more ECG waveforms. In these instances, one of the first electrode 302A and the second electrode 302B acts as the reference electrode discussed above, while the other acts as a ground electrode. The ground electrode may be used by the ECG measurement circuitry 306 to connect the user's body to the ground of the ECG measurement circuitry 306. Accordingly, in these instances the ground electrode is also positioned to contact the user's skin when the electronic device is releasably coupled to the user's body.

In other variations, one of the first electrode 302A and second electrode 302B is not used by the ECG measurement circuitry 306 in generating an ECG waveform. For example, the first electrode 302A may be utilized by the capacitance measurement circuitry 304 but not by the ECG measurement circuitry 306. In some of these variations, the hybrid sensor 300 may include an additional electrode (not shown) that is connected to and used by the ECG measurement circuitry 306 as a reference or ground electrode, but is not used by the capacitance measurement circuitry 304 to calculate a perspiration metric. In instances where an electrode may be selectively connected to either the ECG measurement circuitry 306 or the capacitance measurement circuitry 304, the hybrid sensor 300 may include a switching arrangement (e.g., utilizing one or more switches), which may be any arrangement of components configured to selectively switch the electrode's connection. In the variation shown in FIG. 3A, the hybrid sensor 300 includes a first switching arrangement 308A that selectively connects the first electrode 302A to either the ECG measurement circuitry 306 or the capacitance measurement circuitry 304, and a second switching arrangement 308B that selectively connects the second electrode 302B to either the ECG measurement circuitry 306 or the capacitance measurement circuitry 304.

FIG. 3B shows an example process flow 310 by which an electronic device including the hybrid sensor 300 may selectively perform ECG and perspiration measurements using the hybrid sensor 300. At step 312, a determination is made (e.g., by a processor of an electronic device incorporating the hybrid sensor 300) whether to initiate a measurement session. The measurement session may be a perspiration measurement session during which a first set of electrodes is used to calculate a perspiration metric, or may be an ECG measurement session during which a second set of electrodes is used to generate one or more ECG waveforms. At least one electrode is included in both the first and second sets of electrodes. For example, in some instances, the first electrode 302A of the plurality of electrodes of hybrid sensor 300 is included in both the first and second sets of electrodes. In some of these variations, the second electrode 302B is also included in both the first and second sets of electrodes.

The decision to initiate a particular measurement system may be based on any suitable criteria such as discussed above. For example, perspiration measurement sessions may be initiated on demand, automatically on a scheduled basis, or automatically when certain criteria are met. For example, in some variations, the perspiration session is initiated while an electronic device is monitoring a user exercise session. In these instances, the electronic device may include an activity tracking application that may track an exercise session, during which a set of sensors monitors a given user activity (e.g., running, walking, cycling, or the like) during an exercise session and tracks a set of exercise metrics associated with the activity (e.g., step count, distance traveled, calories burned, combinations thereof, or the like). Monitoring of the exercise session may begin automatically in response to determining that a user is engaging in a predetermined activity, or may begin in response to a user input signaling the start of an exercise session. In some instances, the electronic device automatically initiates a perspiration measurement session in response to the electronic device monitoring an exercise session (e.g., either immediately when the electronic device begins monitoring the exercise session or after a set of criteria associated with the exercise session is met). The electronic device may optionally generate a user interface (e.g., via the activity tracking application) while tracking an exercise session, and this user interface may display one or more attributes of the workout. In some instances, this user interface may display a perspiration metric during the exercise session.

Similarly ECG measurement sessions may be initiated on demand or automatically. Because an ECG measurement session may require that a user actively contact the third electrode 302C, ECG measurement sessions may, in these instances, only be automatically initiated when a user is actively contacting the second set of electrodes. In some instances, an ECG measurement session may interrupt a perspiration measurement session, or vice versa. For example, if the electronic device is performing a perspiration measurement session while the electronic device is tracking an exercise session, the electronic device may stop the perspiration measurement session and initiate an ECG measurement session in response to a user requesting that the electronic device perform an ECG measurement during the exercise session. In some of these variations, the electronic device may automatically resume the perspiration measurement session after the ECG measurement session has been completed.

If a perspiration measurement session is selected at step 312, the first set of electrodes is connected to the capacitance measurement circuitry 304 at step 314. The first set of electrodes includes at least the first electrode 302A and the second electrode 302B. At step 316, the capacitance measurement circuitry 306 will perform a set of capacitance measurements using the first set of electrodes as described previously, which is used to calculate a perspiration metric. At step 318, a determination is made whether the perspiration measurement session is complete. If the session is not complete, the process returns to step 316 to perform additional measurements and calculate additional values of one or more perspiration metrics. If the session is complete, the process returns to step 312.

Similarly, if an ECG measurement session is selected at step 312, the second set of electrodes is connected to the ECG measurement circuitry 306 at step 320. The second set of electrodes may include at least the third electrode 302C and one or both of the first and second electrodes 302A, 302B, and in some instances may further include additional electrodes. As step 322, the ECG measurement circuitry 306 performs an ECG measurement using the second set of electrodes to generate an ECG waveform as discussed above. At step 324, a determination is made whether the ECG measurement session is complete. If the session is not complete, the process returns to step 322 to perform additional measurements and generate additional ECG waveforms. If the session is complete, the process returns to step 312.

When the perspiration sensors described herein are used to calculate a perspiration metric, the external environment may impact the accuracy of the measurements performed by the perspiration sensors. For example, depending on the configuration of the electronic device incorporating the perspiration sensor and how the electronic device is coupled to the user, parts of the electrodes of a given perspiration sensor may be exposed to the ambient air. Depending on the relative temperature and humidity of the ambient air, sweat near the exposed parts of the electrodes may secrete and evaporate at different rates and thereby impact the capacitance values measured by a capacitance measurement circuit. This impact may be even greater at higher sweat rates. Accordingly, the devices described may be configured to reduce the impact of the external environment on measurements.

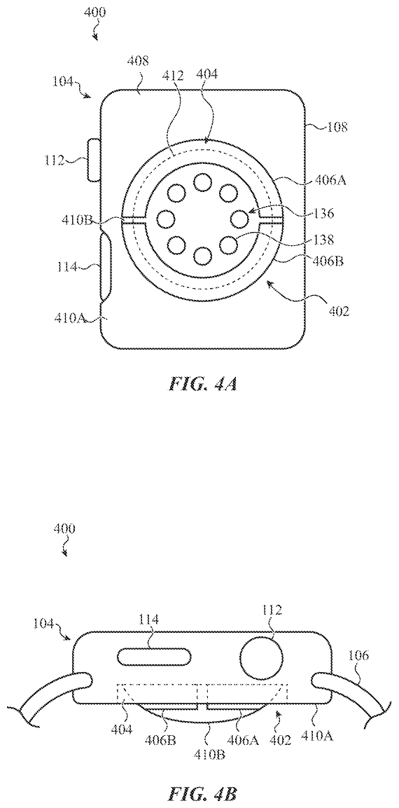

For example, in some variations an electronic device may be configured to define a cavity that houses a portion of the electrodes of a given perspiration sensor. For example, FIGS. 4A and 4B show rear and side views, respectively, of a variation of an electronic device 400 having a perspiration sensor 402 and that defines a cavity 404. The electronic device 400 may otherwise be configured as described above with respect to the wearable electronic device 100 of FIGS. 1A-1D (with like components using the same figure labels). For example, the electronic device 400 may include a watch body 104 having a display (not shown), a crown 112, a button 114, and an optical sensor 136 including a plurality of windows 138 (not shown in FIG. 4B for sake of illustration), and the watch body 104 may be releasably coupled to a user using a strap 106.

The perspiration sensor 402 includes a first electrode 406A and a second electrode 406B, which may be used to perform perspiration measurements as discussed previously. The first electrode 406A and the second electrode 406B are positioned on (and thereby define a portion of) a skin-facing exterior surface 408 of the wearable electronic device 100. The cavity 404 may be defined in the skin-facing exterior surface 408 of the electronic device 400. When the skin-facing exterior surface 408 is positioned against the skin of the user (e.g., when the electronic device 400 is releasably coupled to the user), the user's skin may at least partially cover and seal the cavity 404, which may thereby shield the cavity 404 from the external environment. At least one of the first and second electrodes 406A and 406B is positioned to extend at least partially into the cavity 404. While both the first and second electrodes 406A, 406B are shown in FIGS. 4A and 4B as extending at least partially into the cavity 404, in other instances only one of the first and the second electrodes 406A, 406B extends at least partially into the cavity 404.

In some variations, one or both of the first and second electrodes 406A, 406B extend only partially into cavity 404, such that another portion of that electrode is positioned outside of the cavity 404. For example, in the variation shown in FIGS. 4A and 4B, the first electrode 406A includes a first portion and a second portion (separated by line 412 in FIG. 4A) where the first portion (shown in phantom in FIG. 4B) is positioned inside the cavity 404 and the second portion is positioned outside of the cavity 404. Similarly, the second electrode 406B includes a first portion and a second portion (also separated by line 412 in FIG. 4A) where the first portion (also shown in phantom in FIG. 4B) is positioned inside the cavity 404 and the second portion is positioned outside of the cavity 404. In instances where one or both of the first and second electrodes 406A, 406B are used to perform an ECG measurement as part of a hybrid sensor (e.g., the hybrid sensor 300 discussed above with respect to FIGS. 3A and 3B), having respective portions of these electrodes positioned outside of the cavity 404 may facilitate contact between the electrodes and a user's skin during ECG measurements.

To define the cavity 404, the skin-facing exterior surface 408 includes a first region 410A surrounding a second region 410B. At least a portion of the second region 410B (i.e., the portion of the second region 410B positioned between the first region 410A and line 412 in FIG. 4A) is recessed relative to the first region 410A to define the cavity 404. In some variations, at least a portion of the second region 410B (i.e., the portion of the second region 410B encircled by line 412) protrudes past the first region 410A. In other words, line 412 represents the portion of the second region 410B that is positioned at the same height as the first region 410A. In the variation shown in FIG. 4B, the first region 410A of the skin-facing exterior surface 408 is a flat region of the housing of the electronic device 400, while the second region 410B has a circular shape having a domed profile. In these instances, the cavity 404 has an annular shape. It should be appreciated that the first and second regions 410A, 410B may have different shapes and profiles that define a cavity having a different shape (e.g., circular, rectangular, or the like). In some variations in which the electronic device 400 includes an optical sensor 138 having a set of windows 138, the set windows 138 of the optical sensor is positioned in the second region such that the first region surrounds the set of windows 138.

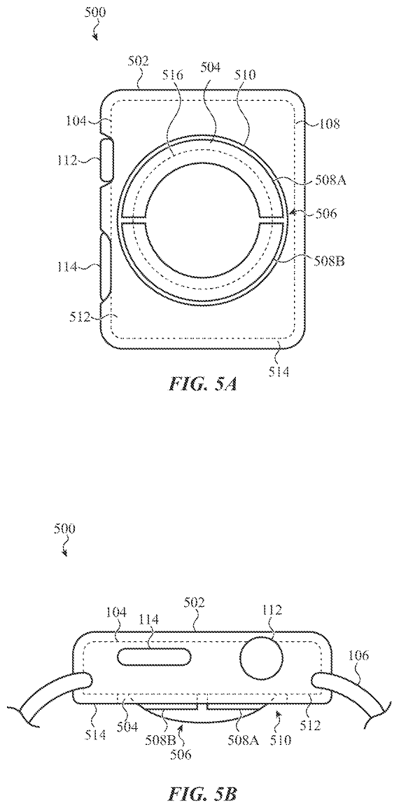

In other variations, an electronic device includes a removable accessory component that may, in conjunction with a housing of the electronic device, define a cavity as discussed above. For example, FIGS. 5A and 5B show rear-facing and side views, respectively, of a variation of a system that includes an electronic device 500 and a removable accessory component 502 that at least partially defines a cavity 504. The electronic device 500 may otherwise be configured as described above with respect to the wearable electronic device 100 of FIGS. 1A-1D (with like components using the same figure labels). For example, the electronic device 500 may include a watch body 104 having a display (not shown), a crown 112, and a button 114, and the watch body 104 may be releasably coupled to a user using a strap 106.

The accessory component 502 is configured to be releasably coupled to a skin-facing surface 512 of a housing of the electronic device 500. In the variation shown in FIGS. 5A and 5B, the accessory component 502 is configured as a case that at least partially encircles a portion of the electronic device 500 (e.g., the watch body 104) to releasably couple the accessory component 502 thereto. In other instances, the accessory component 502 may include a plate or other body that may be placed against the skin-facing surface 512 and releasably coupled thereto (e.g., using magnets, mechanical fasters, or the like). In this way, a user may decide to releasably couple the accessory component 502 to the electronic device 500 to define the cavity 504. The cavity 504 may shield a perspiration sensor 506 during perspiration measurements, such as described above with respect to the cavity 404 of FIGS. 4A and 4B.

Specifically, the accessory component 502 defines an opening 510 that at least partially defines the cavity 504. When the accessory component 502 is releasably coupled to the electronic device 500 the opening 510 is positioned such that at least a portion of the skin-facing surface 512 of the electronic device 500 is exposed through the opening 510. The perspiration sensor 506, which may be configured in any suitable manner as described previously, includes a first electrode 508A and a second electrode 508B, and the opening 510 is positioned such that at least a portion of each of the first electrode 508A and the second electrode 508B are exposed through the opening 510, thereby allowing the perspiration sensor 506 to perform perspiration measurements as discussed above.

In some variations, a portion of the skin-facing surface 512 of the electronic device 500 extends at least partially through the opening 510. For example, in the variation shown in FIGS. 5A and 5B, a portion of the electronic device 500 extends through the opening 510 and protrudes past a skin-facing surface 514 of the accessory component 502. In these instances, a first portion of the electronic device 500 (e.g., positioned between the perimeter of the opening 510 and line 516 in FIG. 5A) is recessed relative to the skin-facing surface 514 of the accessory component 502, which defines, along with the accessory component 502, the cavity 504. Additionally, a second portion of the electronic device 500 (e.g., positioned within line 516) protrudes past the skin-facing surface 514 of the accessory component 502. In these variations, line 516 represents the portion of the skin-facing surface 512 of the housing that is positioned at the same height as the skin-facing surface 514 of the accessory component 502. While the cavity 504 has an annular shape in the variation shown in FIGS. 5A and 5B, the cavity 504 may be defined with any suitable shape such as described above.

In some instances, at least a portion of one or both of the first and second electrodes 508A, 508B is positioned within the cavity 504 when the accessory device 502 is releasably coupled to the electronic device 500. Additionally or alternatively, at least a portion of one or both of the first and second electrodes 508A, 508B are positioned outside the end of the cavity 504 when the accessory device 502 is releasably coupled to the electronic device 500. In the variation shown in FIGS. 5A and 5B, the first electrode 508A includes a first portion and a second portion (separated by line 516 in FIG. 5A) where the first portion is positioned inside the cavity 504 and the second portion is positioned outside of the cavity 504. Additionally or alternatively, the second electrode 508B includes a first portion and a second portion (also separated by line 516 in FIG. 5A) where the first portion is positioned inside the cavity 504 and the second portion is positioned outside of the cavity 504. In instances where one or both of the first and second electrodes 508A, 508B are used to perform an ECG measurement as part of a hybrid sensor (e.g., the hybrid sensor 300 discussed above with respect to FIGS. 3A and 3B), having respective portions of these electrodes positioned outside of the cavity 504 may facilitate contact between the electrodes and a user's skin during ECG measurements.

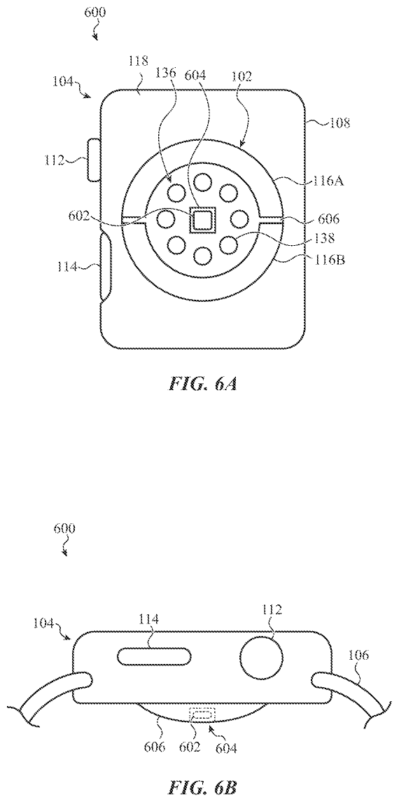

While the variations of electronic devices described above with respect to FIGS. 1A-5B each are shown as having a single perspiration sensor, in some instances an electronic device (which may otherwise be configured in any manner as described above with respect to FIGS. 1A-5B) may include a plurality of perspiration sensors. FIGS. 6A and 6B show one such variation of an electronic device 600 including multiple perspiration sensors. The electronic device 600 may otherwise be configured as described above with respect to the wearable electronic device 100 of FIGS. 1A-1D (with like components using the same figure labels). For example, the electronic device 600 may include a watch body 104 having a display (not shown), a crown 112, a button 114, and an optical sensor 136 including a plurality of windows 138 (not shown in FIG. 6B for the sake of illustration), and the watch body 104 may be releasably coupled to a user using a strap 106.

The electronic device 600 includes first and second perspiration sensors. In the variation shown in FIGS. 6A and 6B, the first perspiration sensor is configured as the perspiration sensor 102 described above with respect to FIGS. 1A-1D, and that includes a first electrode 116A and a second electrode 116B (not depicted in FIG. 6B) that are positioned on (and thereby define) a portion of a skin-facing surface 118 of a housing 108 of the electronic device 600. The second perspiration sensor 602 is contained within a cavity 604 in the skin-facing surface 118 of the housing 108. The second perspiration sensor 604 may comprise any sensor capable of outputting a signal indicative of the amount of moisture present in the cavity 604 (e.g., a humidity sensor, or the like). When the electronic device 600 is releasably coupled to a user such that the skin-facing surface 118 contacts the skin of a user, the user's skin may seal the cavity 604.

The second perspiration sensor 602 and cavity 604 may be positioned closer to a center of the skin-facing surface 118 of the housing 108 than one or both of the electrodes of the first perspiration sensor 102. The second perspiration sensor 602 may be relatively small when compared to the first perspiration sensor 102, and may produce signals having a lower signal to noise ratio as compared to measurements performed by the first perspiration sensor 102. By positioning the second perspiration sensor 602 and cavity closer to the center of the skin-facing surface 118, it may be better shielded relative to the external environment than the electrodes of the first perspiration sensor 102. For example, in instances where the skin-facing surface 118 of the housing 108 includes a region 606 having a domed profile as discussed above, defining the cavity in the region 606 (e.g., in a center of the region 606) may increase the likelihood that the user's skin will fully seal the cavity 604 when the electronic device 600 is releasably coupled to a user.

In the variation shown in FIG. 6A, first electrode 116A and second electrode 116B of the perspiration sensor 102 each have a semi-annular shape, and the cavity 604 (and thereby the second perspiration sensor 602) is positioned between at least a portion of the first electrode 116A and at least a portion of the second electrode 116B of the perspiration sensor 102. Additionally or alternatively, in variations where one or both of the first electrode 116A and second electrode 116B are positioned to extend at least partially into an annular cavity defined in the skin-facing surface (such as some of the embodiments of the electronic device 400 and cavity 404) described above with respect to FIGS. 4A and 4B), this annular cavity may encircle the cavity 604 that encloses the perspiration sensor 602 (i.e., the second perspiration sensor 602 does not extend outside of the cavity 604). Additionally or alternatively, in instances where the electronic device 600 includes an optical sensor 136 that has a set of windows 138 (not depicted in FIG. 6B), some or all of the windows may be positioned between the cavity 604 (and with it, the second perspiration sensor 602) and one or both of the first and second electrodes 116A, 116B.

The electronic device 600 may use measurements from both the first and second perspiration sensors in calculating a perspiration metric. For example, when the method 200 described above with respect to FIG. 2 is performed by a device having a plurality of perspiration sensors (such as electronic device 600 of FIGS. 6A and 6B), the method may further comprise obtaining a set of additional measurements using a second perspiration sensor. In these instances, at step 206 the method includes calculating the perspiration metric using both the set of capacitance measurements obtained by a first perspiration sensor and the set of additional measurements obtained by the second perspiration sensor. As an example, a relationship between a measured capacitance and an estimated sweat rate may also utilize information from the set of additional measurements as an input.

As described above, one aspect of the present technology involves measuring physiological signals, such as perspiration and ECG measurements. The present disclosure contemplates that in some instances this gathered data may include personal information data that uniquely identifies or can be used to contact or locate a specific person. Such personal information data can include demographic data, location-based data, telephone numbers, email addresses, Twitter IDs (or other social media aliases or handles), home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining the privacy and security of personal information data. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and revised to adhere to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method for measuring physiological signals using an electronic device comprising a plurality of electrodes, the method comprising:
during a perspiration measurement session:
connecting a first set of electrodes of the plurality of electrodes to capacitive measurement circuitry;
performing a set of capacitance measurements using the first set of electrodes; and
calculating a perspiration metric using the set of capacitance measurements; and
during an electrocardiogram measurement;
connecting a second set of electrodes of the plurality of electrodes to electrocardiogram measurement circuitry; and
performing an electrocardiogram measurement to generate an ECG waveform; wherein:
the plurality of electrodes comprises a first electrode and a second electrode;
the first set of electrodes comprises the first electrode and the second electrode; and
the second set of electrodes comprises the first electrode.

2. The method of claim 1, wherein:
the second set of electrodes comprises the second electrode.

3. The method of claim 2, wherein:
the plurality of electrodes comprises a third electrode; and
the second set of electrodes comprises the third electrode.

4. The method of claim 1, wherein the perspiration metric is an estimated sweat rate.

5. The method of claim 1, wherein the perspiration measurement session is initiated while the electronic device is tracking an exercise session.

6. The method of claim 5, wherein the perspiration measurement session is initiated automatically in response to the electronic device tracking the exercise session.

7. A device for calculating a perspiration metric of a user, comprising:
a housing having an exterior surface;
a perspiration sensor comprising:
a first electrode; and
a second electrode; and
a processer configured to calculate the perspiration metric using the perspiration sensor; wherein:
the exterior surface of the housing defines a cavity; and
the first electrode extends at least partially into the cavity.

8. The device of claim 7, wherein:
the second electrode extends at least partially into the cavity.

9. The device of claim 7, wherein:
a portion of the first electrode is positioned outside of the cavity.

10. The device of claim 7, wherein:
the exterior surface comprises a first region surrounding a second region; and
a first portion of the second region is recessed relative to the second region to define the cavity.

11. The device of claim 10, wherein:
a second portion of the second region protrudes past the first region.

12. The device of claim 10, wherein:
the cavity has an annular shape.

13. The device of claim 10, further comprising:
an optical sensor comprising a set of windows; wherein the set of windows is positioned in the second region.

14. The device of claim 7, wherein:
the perspiration sensor is a first perspiration sensor; and
the device comprises a second perspiration sensor.

15. The device of claim 7, wherein:
the perspiration sensor comprises capacitance measurement circuitry configured to measure a capacitance between the first electrode and the second electrode.

16. A device for calculating a perspiration metric of a user, comprising:
a housing having an exterior surface that defines a first cavity;
a first perspiration sensor comprising:
a first electrode defining a first portion of the exterior surface; and
a second electrode defining a second portion of the exterior surface;
a second perspiration sensor contained within the first cavity; and
a processer configured to calculate the perspiration metric using both the first perspiration sensor and the second perspiration sensor.

17. The device of claim 16, wherein:
the housing defines a second cavity; and
the first electrode extends at least partially into the second cavity.

18. The device of claim 17, wherein:
the second cavity encircles the first cavity.

19. The device of claim 16, wherein:
the first cavity is positioned between at least a portion of the first electrode and at least a portion of the second electrode.

20. The device of claim 16, wherein:
the first cavity is positioned closer to a center of the exterior surface of the housing relative to the first electrode.
